(19) United States
(12) Patent Application Publication
(10) Pub. No.: US 2024/0050309 A1
(43) Pub. Date: Feb. 15, 2024

(54) ATOMIZATION DEVICE FOR FACIAL STEAMER

(71) Applicant: Shenzhen Aifan Technology Co., Ltd., Shenzhen (CN)

(72) Inventor: Jianping LIU, Shenzhen (CN)

(73) Assignee: Shenzhen Aifan Technology Co., Ltd., Shenzhen (CN)

(21) Appl. No.: 18/307,982

(22) Filed: Apr. 27, 2023

(30) Foreign Application Priority Data
Aug. 9, 2022 (CN) 202222094793.0

(51) Int. Cl. A61H 33/12; A61H 33/06; A61H 33/00; A61M 11/00

(52) U.S. Cl. CPC A61H 33/12; A61H 33/063; A61H 33/6021; A61M 11/005; A61H 2205/022; A61H 2033/068; A61H 2033/0054

(57) ABSTRACT

The present disclosure provides an atomization device for a facial steamer, including a body, an atomization sheet, a heating mechanism, and an atomizing nozzle; the body is provided with a water inlet pipeline, a water outlet, and an inlet pressure balance channel; the heating mechanism is connected with the water outlet of the body; the atomization sheet is arranged between the water outlet and the heating mechanism; the atomizing nozzle is connected with a free end of the heating mechanism; the inlet pressure balance channel is communicated with the heating mechanism; a water storage cavity is formed in the body; and the water storage cavity is located on a balance path of the inlet pressure balance channel.

### CROSS-REFERENCE TO RELATED APPLICATIONS

The application claims priority to Chinese patent application No. 202222094793.0, filed on Aug. 9, 2022, the entire contents of which are incorporated herein by reference.

### TECHNICAL FIELD

The present disclosure belongs to the technical field of facial steamers, in particular to, an atomization device for a facial steamer.

### BACKGROUND

At present, facial steamers on the market use Positive Temperature Coefficient (PTC) heating to boil water to a temperature exceeding its boiling point, so that the water turns into gas, which changes the physical structure of water. Due to a high temperature, it is easy to scald a user if a distance is not well controlled. Moreover, after long-term use of this kind of facial steamer, a negative pressure is easily generated inside the atomization device, resulting in a poor atomization effect.

According to the Chinese patent document No. 202023346379.1, an atomization device capable of automatically adjusting an air pressure is disclosed. The atomization device mainly includes a water conveying channel, which includes a water inlet and a water outlet; an atomizing sheet, which is arranged at the water outlet to atomize water; a heating mechanism, which is connected to the water outlet to heat water mist and controlling a temperature of the water mist to be between 45 DEG C. and 60 DEG C.; and an inlet pressure balance channel, which is arranged on the water conveying channel to balance internal and external air pressures of the atomization device. Liquid water is atomized through high-frequency resonance of the atomizing sheet, and then the water mist is heated to control the temperature of the water mist at 45 to 60 DEG C. An internal air pressure balance can be automatically adjusted, making a use effect more stable and effective. After atomization, part of the water mist enters the inlet pressure balance channel and is quickly condensed back into water droplets or a water curtain without being heated. As a result, the inlet pressure balance channel cannot function as expected.

Based on the above situation, the applicant further optimizes the inlet pressure balance channel on the basis of the disclosed patent literature to solve the above problem.

### SUMMARY

In order to achieve the above objective, the present disclosure adopts the following technical solution:

The present disclosure provides an atomization device for a facial steamer, including a body, an atomization sheet, a heating mechanism, and an atomizing nozzle; the body is provided with a water inlet pipeline, a water outlet, and an inlet pressure balance channel; the heating mechanism is connected with the water outlet of the body; the atomization sheet is arranged between the water outlet and the heating mechanism; the atomizing nozzle is connected with a free end of the heating mechanism; the inlet pressure balance channel is communicated with the heating mechanism; a water storage cavity is formed in the body; and the water storage cavity is located on a balance path of the inlet pressure balance channel.

In an improvement of the present disclosure, the water inlet pipeline is provided with a water level detection mechanism.

In an improvement of the present disclosure, the water level detection mechanism is a water level probe.

In an improvement of the present disclosure, the water inlet pipeline is sleeved with a waterproof silica gel.

In an improvement of the present disclosure, the atomization sheet is an ultrasonic atomization sheet.

In an improvement of the present disclosure, the heating mechanism includes a support, a heating source, and a heating tube; the heating source is connected with the support; and the heating tube is arranged in the heating source.

In an improvement of the present disclosure, the heating source is a heating film, a heating wire, or a ceramic heating tube.

In an improvement of the present disclosure, the heating tube is a metal tube or a ceramic tube.

In an improvement of the present disclosure, a gasket is also arranged between the heating mechanism and the atomization sheet.

Compared with the prior art, the present disclosure has the beneficial effects:

An atomization device for a facial steamer provided by the present disclosure includes a body, an atomization sheet, a heating mechanism, and an atomizing nozzle; the body is provided with a water inlet pipeline, a water outlet, and an inlet pressure balance channel; the heating mechanism is connected with the water outlet of the body; the atomization sheet is arranged between the water outlet and the heating mechanism; the atomizing nozzle is connected with a free end of the heating mechanism; the inlet pressure balance channel is communicated with the heating mechanism; a water storage cavity is formed in the body; and the water storage cavity is located on an inlet pressure balance channel path of the inlet pressure balance channel. By the adoption of the above structure, the water storage cavity is formed in the body to store water that is condensed by water mist entering the inlet pressure balance channel, to ensure that the balance path is smooth, so that the inlet pressure balance channel is communicated with the heating to better automatically adjust an internal air pressure balance. The atomization device for the facial steamer has the advantages of reasonable design, simple structure, and more stable use effect.

### BRIEF DESCRIPTION OF THE DRAWINGS

FIG. 1 is a schematic diagram of an exploded structure of an atomization device for a facial steamer in one direction according to the present disclosure.

FIG. 2 is a schematic diagram of a sectional structure of an atomization device for a facial steamer in one direction according to the present disclosure.

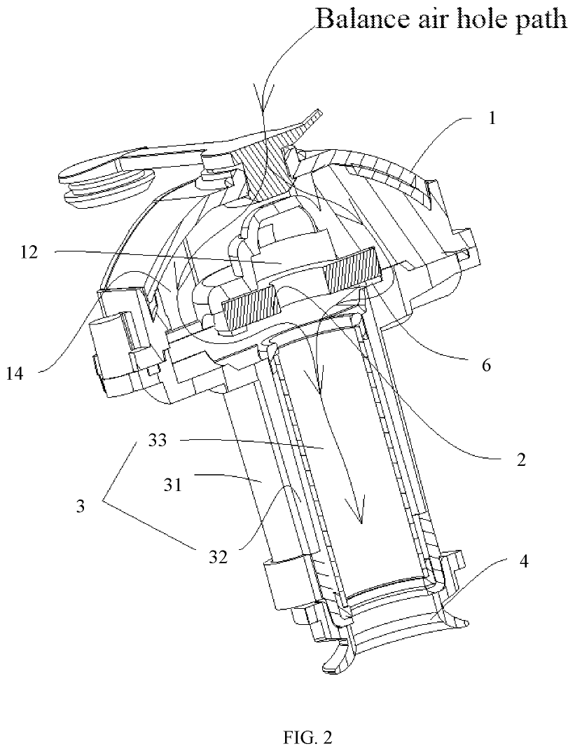

FIG. 3 is a schematic diagram of a sectional structure of an atomization device for a facial steamer in another direction according to the present disclosure.

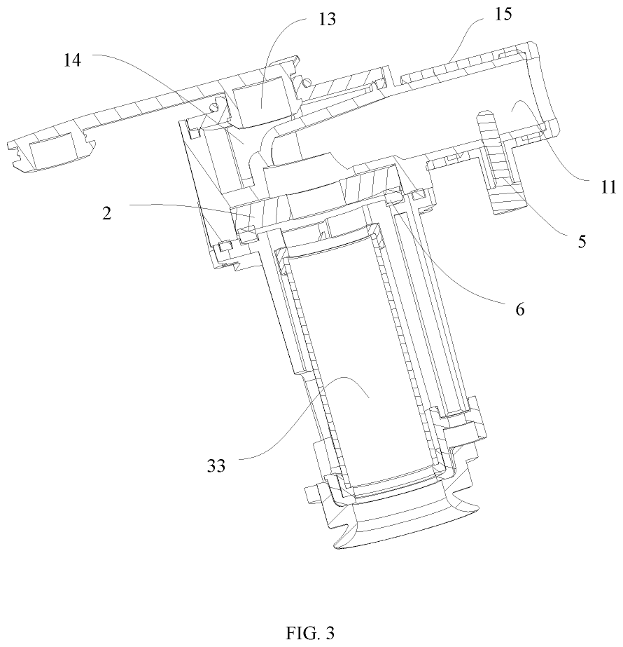

FIG. 4 is a schematic diagram of a three-dimensional structure of an atomization device for a facial steamer in one direction according to the present disclosure.

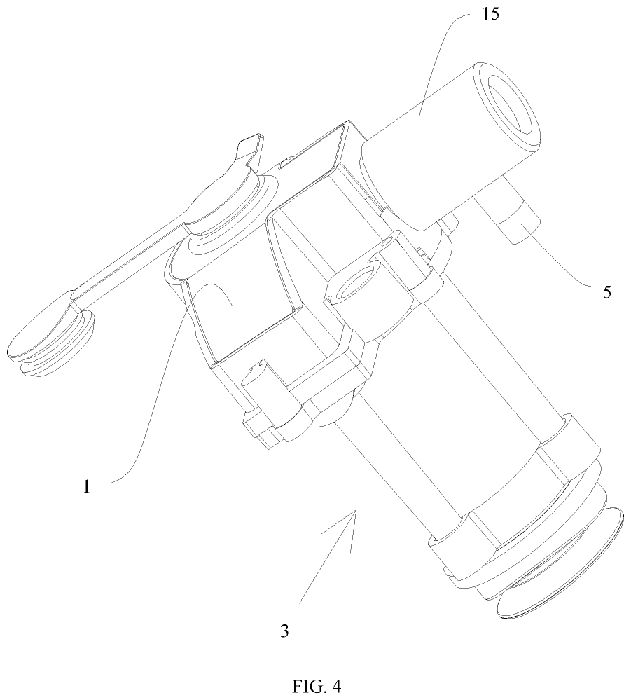

FIG. 5 is a schematic diagram of a three-dimensional structure of an atomization device for a facial steamer in another direction according to the present disclosure.

The implementation, function characteristics and advantages of the present disclosure will be further described in combination with embodiments and with reference to accompanying drawings.

### DETAILED DESCRIPTION OF THE EMBODIMENTS

In the description of the present disclosure, it should be understood that orientations or positional relationships indicated by the terms “center”, “upper”, “lower”, “front”, “rear”. “left”, “right” and the like are orientations or positional relationships as shown in the drawings, and are only for the purpose of facilitating and simplifying the description of the present disclosure instead of indicating or implying that devices or components indicated must have particular orientations, and be constructed and operated in the particular orientations, so that these terms are not construed as limiting the present disclosure In addition, the terms “first” and “second” are only for the purpose of description, and may not be understood as indicating or implying the relative importance.

In the description of the present disclosure, it should be noted that unless otherwise explicitly defined and regulated, the terms “mount”, “connect”, and “couple” shall be understood broadly, and may be, for example, fixedly connected, or detachably connected, or integrally connected, or mechanically connected, or electrically connected, or directly connected, or indirectly connected through an intermediate medium, or interconnection between two components. Those of ordinary skill in the art can understand the specific meanings of the above terms in the present disclosure according to specific situations.

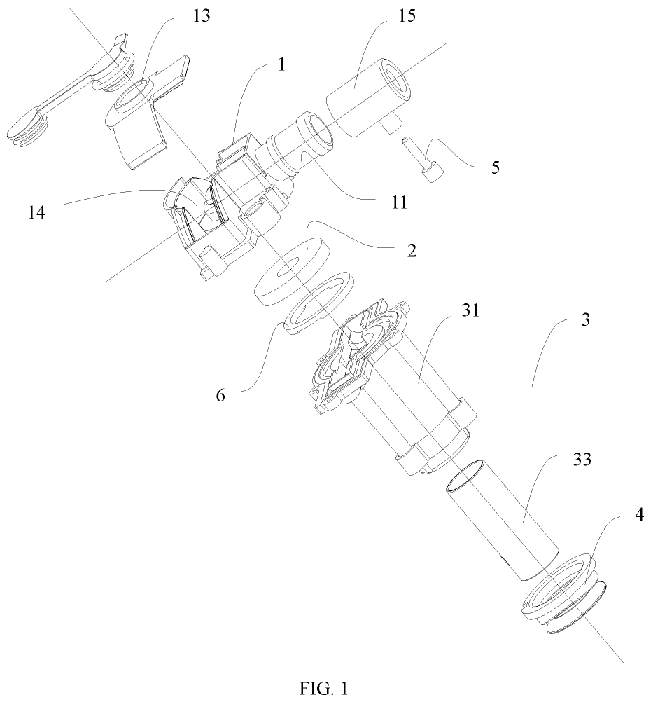

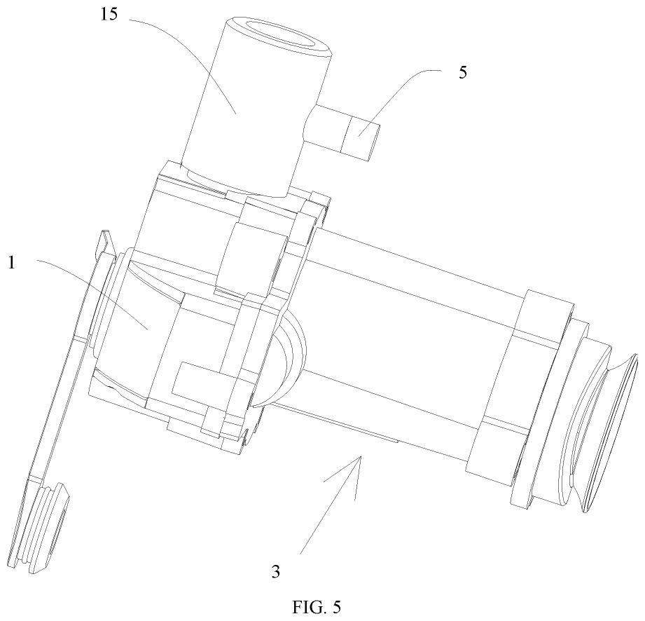

As shown in FIG. 1 and FIG. 5, the present disclosure provides an atomization device for a facial steamer, including a body 1, an atomization sheet 2, a heating mechanism 3, and an atomizing nozzle 4. The body 1 is provided with a water inlet pipeline 11, a water outlet 12, and an inlet pressure balance channel 13. The heating mechanism 3 is connected with the water outlet 12 of the body 1. The atomization sheet 2 is arranged between the water outlet 12 and the heating mechanism 3. The atomizing nozzle 4 is connected with a free end of the heating mechanism 3. The inlet pressure balance channel 13 is communicated with the heating mechanism 3. A water storage cavity 14 is formed in the body 1. The water storage cavity 14 is located on a balance path of the inlet pressure balance channel 13.

Specifically, the balance path means that the water storage cavity 14 is provided, according to a predetermined direction, with two through slots leading to the heating mechanism 3. The inlet pressure balance channel 13 is led to the heating mechanism 3 through the water storage cavity 14 and the two through slots in the water storage cavity.

Specifically, the atomization sheet 2 adopts an ultrasonic atomization sheet. The water inlet pipeline 11 is connected with an external water supply device. A water source enters the atomization sheet 2 from the water inlet pipeline 11. The atomization sheet 2 scatters liquid water molecules to produce naturally flowing water mist. Most water mist is sprayed from the atomizing nozzle 4 via the heating mechanism 3, and a little of the water mist enters the water storage cavity 14 along the balance path and is re-condensed into water droplets in the water storage cavity 14. Some re-condensed water can be collected through the water storage cavity 14, to prevent the water droplets or a water curtain formed in the balance path from blocking the inlet pressure balance channel. After the device is used for some time, a user can shake the device to throw out the water in the water storage cavity. In the present disclosure, the ultrasonic atomization sheet is preferred. The ultrasonic atomization sheet is to use high-frequency electronic oscillation (an oscillation frequency is 1.7 MHz or 2.4 MHz, which is beyond a hearing range of human, and the electronic oscillation is absolutely harmless to human and animals) to scatter the liquid water molecules through the high-frequency resonance of the atomization sheet 2 to produce the naturally flowing water mist. The heating mechanism 3 heats the water mist. During heating, a temperature of the water mist is controlled between 45 DEG C. and 60 DEG C. It is a temperature range that is acceptable to the human skin, makes a user feel comfortable, and will not scald the user.

During working of the heating mechanism 3, a negative pressure is easily generated in the heating mechanism 3, which greatly reduces the atomization effect. The inlet pressure balance channel 13 is arranged on a side wall of the body to communicate the outside to the heating mechanism 3. Air gets in and out from the inlet pressure balance channel 13, which can balance an internal air pressure of the heating mechanism 3 and ensure normal atomization.

When not used, a rubber plug can be arranged on the inlet pressure balance channel 13, to avoid dust from entering the facial steamer from the inlet pressure balance channel 13. When used, the rubber plug is pulled up. Since the internal air rushes out, the dust will not enter the facial steamer from the inlet pressure balance channel 13.

In some embodiments, the water inlet pipeline 11 is provided with a water level detection mechanism 5. The water level detection mechanism 5 is a water level probe. The water level detection mechanism 5 is used for detecting a remaining amount of the water in the body. Specifically, the water inlet pipeline 11 is connected with an external water supply bucket, and supplies water through the external water supply bucket. The water enters the atomization sheet 2 from the water inlet pipeline 11, and is atomized through the atomization sheet 2. When a water level is lower than the water level probe, an alarm is sounded, and the user can stop atomization or add water to the water bucket.

In some embodiments, to prevent untight connection between the external water supply bucket and the water inlet pipeline 11 and prevent the water from overflowing out of the body 1, the water inlet pipeline 11 is sleeved with waterproof silica gel 15.

In some embodiments, the heating mechanism 3 includes a support 31, a heating source 32, and a heating tube 33. The heating source 32 is connected with the support 31. The heating tube (33) is arranged in the heating source 32.

In some embodiments, the heating source 32 may be a heating film, a heating wire, or a ceramic heating tube, or made of other heating materials. The heating source 32 is energy-saving, environmentally-friendly, and low-carbon. The facial steamer on the market has service power of 200-300 W, which has high power consumption. The facial steamer using this embodiment has power consumption of 20 W or below, which saves energy by 90%.

In some embodiments, the heating tube 33 is a metal tube or a ceramic tube. It can be a cylinder, or a metal piece in other regular shapes, for example, a cuboid, a cube, or a cone. It should be noted that the heating tube 33 is certainly hollow, so that the water mist will not be blocked when passing through the heating tube 33, and can be sprayed from the nozzle smoothly. In this embodiment, the heating tube 33 has a length of 20-30 mm and has a diameter of about 10 mm. In this way, the volume of the facial steamer can be controlled, and it is convenient to carry and use the facial steamer.

In some embodiments, a gasket 6 is also arranged between the heating mechanism 3 and the atomization sheet 2. The gasket 6 is used for fixing the atomization sheet 2.

In conclusion, the atomization device for the facial steamer provided by the present disclosure includes a body 1, an atomization sheet 2, a heating mechanism 3, and an atomizing nozzle 4. The body 1 is provided with a water inlet pipeline 11, a water outlet 12, and an inlet pressure balance channel 13. The heating mechanism 3 is connected with the water outlet 12 of the body 1. The atomization sheet 2 is arranged between the water outlet 12 and the heating mechanism 3. The atomizing nozzle 4 is connected with a free end of the heating mechanism 3. The inlet pressure balance channel 13 is communicated with the heating mechanism 3. The water storage cavity 14 is formed in the body 1. The water storage cavity 14 is located on a balance path of the inlet pressure balance channel 13. By the adoption of the above structure, the water storage cavity is formed in the body to store water that is condensed by water mist entering the inlet pressure balance channel, to ensure that the balance path is smooth, so that the inlet pressure balance channel is communicated with the heating to better automatically adjust an internal air pressure balance. The atomization device for the facial steamer has the advantages of reasonable design, simple structure, and more stable use effect.

It can be understood that for those skilled in the art, equivalent substitutions or changes can be made according to the technical solution of the present disclosure and a utility model concept of the technical solution, and all these changes or substitutions shall fall within the protection scope of the claims attached to the present disclosure.

What is claimed is:

1. An atomization device for a facial steamer, comprising a body (1), an atomization sheet (2), a heating mechanism (3), and an atomizing nozzle (4), wherein the body (1) is provided with a water inlet pipeline (11), a water outlet (12), and an inlet pressure balance channel (13); the heating mechanism 43) is connected with the water outlet (12) of the body (1); the atomization sheet (2) is arranged between the water outlet (12) and the heating mechanism (3); the atomizing nozzle (4) is connected with a free end of the heating mechanism (3); the inlet pressure balance channel (13) is communicated with the heating mechanism (3); a water storage cavity (14) is formed in the body (1); and the water storage cavity (14) is located on a balance path of the inlet pressure balance channel (13).

2. The atomization device according to claim 1, wherein the water inlet pipeline (11) is provided with a water level detection mechanism (5).

3. The atomization device according to claim 2, wherein the water level detection mechanism (5) is a water level probe.

4. The atomization device according to claim 1, wherein the water inlet pipeline (11) is sleeved with a waterproof silica gel (15).

5. The atomization device according to claim 1, wherein the atomization sheet (2) is an ultrasonic atomization sheet.

6. The atomization device according to claim 1, wherein the heating mechanism (3) comprises a support (31), a heating source (32), and a heating tube (33); the heating source (32) is connected with the support (31); and the heating tube (33) is arranged in the heating source (32).

7. The atomization device according to claim 6, wherein the heating source (32) is a heating film, a heating wire, or a ceramic heating tube.

8. The atomization device according to claim 6, wherein the heating tube (33) is a metal tube or a ceramic heating tube.

9. The atomization device according to claim 6, wherein a gasket (6) is also arranged between the heating mechanism (3) and the atomization sheet (2).
